(19) United States
(12) Patent Application Publication
(10) Pub. No.: US 2026/0285805 A1
(43) Pub. Date: Sep. 24, 2026

(54) PROCESS FOR PRODUCING ISOPHORONE NITRILE

(71) Applicant: Evonik Operations GmbH, Essen (DE)

(72) Inventors: Jan Benedikt Metternich, Recklinghausen (DE); Jörg-Joachim Nitz, Essen (DE); Matthias Mendorf, Dortmund (DE); Thomas Müller, Altenstadt (DE); Bernd Glöckler, Bruchköbel (DE); Christian John, Herne (DE)

(73) Assignee: Evonik Operations GmbH, Essen (DE)

(21) Appl. No.: 19/573,010

(22) Filed: Mar. 20, 2026

(30) Foreign Application Priority Data
Mar. 21, 2025 (EP) 25165275.6

(51) Int. Cl. C07C 255/46; C07C 45/45; C07C 49/543; C07C 253/10; C07C 253/34

(52) U.S. Cl. CPC C07C 255/46; C07C 45/45; C07C 49/543; C07C 253/10; C07C 253/34

(57) ABSTRACT

The present disclosure provides a process for producing isophorone nitrile comprising reacting a mixture comprising the valuable substances a) isophorone and b) at least one keto- and optionally hydroxyl-containing hydrocarbon having a molecular weight of 178-276 g/mol with hydrogen cyanide to afford isophorone nitrile. In some embodiments, the mixture includes at least two different keto- and optionally hydroxyl-containing hydrocarbons having a molecular weight of 178-276 g/mol.

The present invention relates to a process for producing isophorone nitrile.

Isophorone nitrile is an important industrial chemical whose relevance is based inter alia on its use as a reactant for the synthesis of isophoronediamine employed as an epoxy hardener and isophorone diisocyanate producible therefrom which is of great relevance to polyurethane chemistry. Processes for producing isophorone nitrile are therefore of great interest. Isophorone nitrile is typically produced from isophorone.

Isophorone may be synthesized from acetone under base catalysis: Thus, GB 733,650 A for example discloses that acetone may be converted into isophorone and higher condensation products under base catalysis. In a preferred embodiment, the higher condensation products are recycled into the reaction mixture.

DE 1 205 525 B discloses that byproducts formed in the condensation of acetone to afford isophorone may be treated with the aqueous alkali metal hydroxide solution at relatively high temperatures to allow recovery of low-boiling substances, especially acetone. The described process is also referred to as hydrolysis.

DE 26 45 281 A1 discloses a process for producing isophorone wherein a mixture of acetone, water and an alkali metal catalyst is reacted in a column and a fraction containing isophorone, water and unconverted acetone is recycled from the bottom of the reaction zone to a hydrolysis zone, from where acetone is recycled into the reaction and isophorone is discharged.

DE 10 2010 062 587 A1 is concerned with the object of improving reduced yields attributable to the formation of undesired byproducts such as diacetone alcohol, mesityl oxide, phorone, mesitylene and overcondensates and teaches subjecting a valuable stream containing isophorone and higher condensates to a hydrolysis in order to effect partial or complete conversion of the more highly condensed products into isophorone, acetone and other valuable products. The valuable stream discharged from the hydrolysis is subjected to a phase separation. Isophorone is isolated from the resulting organic phase and purified by customary methods.

CN 103435461 B discloses a process for producing isophorone from acetone wherein acetone is reacted in the presence of an alkaline catalyst to afford a product mixture comprising water, mesityl oxide, isopropylidene isomers, mesitylene, isophorone and tetramerization products. After the separation and recirculation of acetone and the separation of the lighter components, the heavy boilers (comprising isophorone and tetramerization products) are purified and isophorone is obtained in a purity of 99%.

CN 102367223 B discloses a process for producing isophorone in which acetone and water are reacted in the presence of a catalyst and the reaction mixture is hydrolysed and subjected to a phase separation. In the hydrolysis high boilers having 12 or more carbon atoms and unreacted acetone are separated from the isophorone. Acetone is then recycled to the reaction.

Processes for producing isophorone nitrile are also described in the prior art:

DE 39 42 371 C2 discloses a process for producing isophorone nitrile comprising addition reaction of hydrogen cyanide onto isophorone in the presence of an alkali metal compound.

U.S. Pat. No. 5,011,968 A discloses a process producing isophorone nitrile wherein isophorone is reacted with hydrogen cyanide in the presence of a quaternary ammonium hydroxide catalyst.

WO 2004/056753 A1 also discloses a process for producing isophorone nitrile where isophorone is reacted with hydrogen cyanide in the presence of a base as catalyst.

In the context of a synthesis of isophoronediamine WO 2012/076317 A1 discloses a process for producing isophorone nitrile comprising initially producing isophorone from acetone by catalysed aldol condensation, working up the reaction product, hydrolyzing the valuable stream and separating it into an organic fraction and an aqueous fraction and obtaining the isophorone from the organic fraction by customary purification processes, preferably by distillation.

A particular disadvantage of the process disclosed in WO 2012/076317 A1 is that the processes employed for purification of isophorone, especially the distillation recited as preferable, are very energy intensive and thus lead to an unfavourable energy balance when the synthesis of isophorone nitrile is viewed holistically. A further disadvantage is that the processes for producing isophorone nitrile known in the prior art lack sufficiently high yields and selectivities. It is accordingly an object of the present invention to improve the energy balance and the yields and selectivities in the synthesis of isophorone nitrile.

This present object is achieved by the process according to the invention for producing isophorone nitrile comprising reacting a mixture comprising the valuable substances
(1) isophorone and
(2) at least one keto- and optionally hydroxyl-containing hydrocarbon having a molecular weight of 178-276 g/mol
   with hydrogen cyanide to afford isophorone nitrile.

The present invention provides a process for producing isophorone nitrile wherein a mixture comprising the valuable substances isophorone and at least one keto- and optionally hydroxyl-containing hydrocarbon having a molecular weight of 178-276 g/mol is reacted with hydrogen cyanide to afford isophorone nitrile. Thus a portion of the produced isophorone nitrile may derive from the reaction of isophorone with hydrogen cyanide (HCN). The produced isophorone nitrile may also derive at least in part from the reaction of the at least one keto- and optionally hydroxyl-containing hydrocarbon having a molecular weight of 178-276 g/mol with hydrogen cyanide (HCN), wherein it has surprisingly been found that the at least one keto- and optionally hydroxyl-containing hydrocarbon can eliminate radicals having a higher molecular weight than isophorone which account for the higher molecular weight than isophorone. It is particularly preferable when the produced isophorone nitrile derives from the reaction of isophorone and from the reaction of at least one keto- and optionally hydroxyl-containing hydrocarbon having a molecular weight of 178-276 g/mol with hydrogen cyanide.

The valuable substance mixture comprises not only isophorone but also at least one keto- and optionally hydroxyl-containing hydrocarbon having a molecular weight of 178-276 g/mol. It is preferable when at least two, more preferably at least three, yet more preferably at least four, very particularly preferably at least five, yet more preferably at least six, yet more preferably at least seven, yet more preferably at least eight and most preferably at least nine different keto- and optionally hydroxyl-containing hydrocarbons having a molecular weight of 178-276 g/mol are present.

It is preferable when at least one of the keto- and optionally hydroxyl-containing hydrocarbons having a molecular weight of 178-276 g/mol has a structure selected from one of the following structural formulae (I) to (IX):

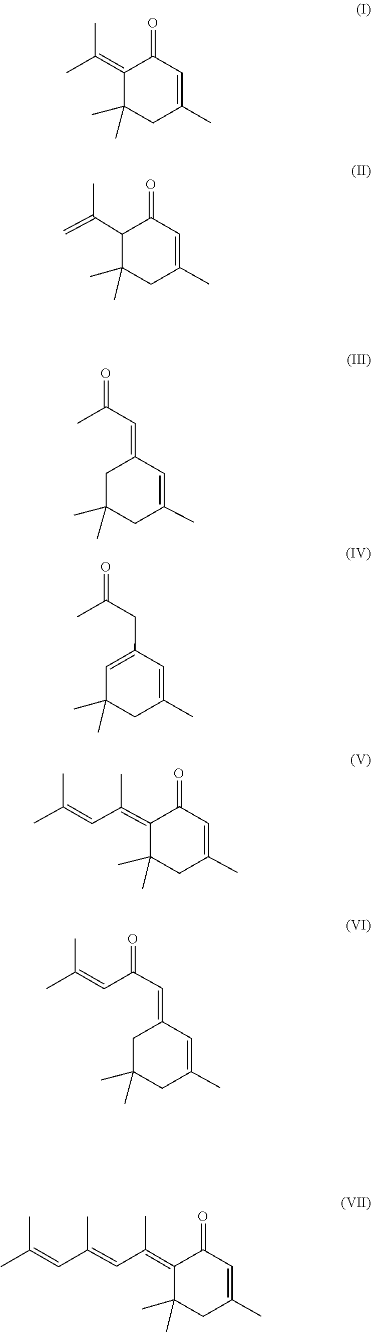

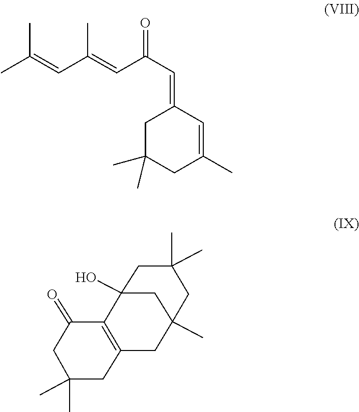

It is preferable when at least two, more preferably at least three, yet more preferably at least four, very particularly preferably at least five, yet more preferably at least six, yet more preferably at least seven, yet more preferably at least eight and most preferably nine different keto- and optionally hydroxyl-containing hydrocarbons having a molecular weight of 178-276 g/mol each have one of the structures selected from the group of structural formulae consisting of (I), (II), (III), (IV), (V), (VI), (VII), (VIII) and (IX).

It is preferable when the mass fraction of keto- and optionally hydroxyl-containing hydrocarbons having a molecular weight of 178-276 g/mol in the valuable substance mixture, based on the total mass of the valuable substance mixture before addition of hydrogen cyanide, is between 1% and 50% by weight, more preferably between 3% and 15% by weight, yet more preferably between 4% and 10% by weight.

The at least one keto- and optionally hydroxyl-containing hydrocarbon having a molecular weight of 178-276 g/mol is preferably at least one overcondensate from the synthesis of isophorone by aldol condensation of acetone. Corresponding overcondensates deriving from the condensation of four or more acetone molecules in the aldol condensation, the formation of isophorone requiring only three acetone molecules, have in the prior art hitherto typically been separated from the target product isophorone by customary fractionation processes, especially by distillation, after hydrolysis, phase separation of the hydrolysate and removal of organic components having a molar mass of <126 g/mol from the organic phase of the phase separation. It is accordingly a significant aspect of the present invention that it was found to be advantageous, contrary to current assumptions for the synthesis of isophorone nitrile from isophorone and hydrogen cyanide, not to remove the overcondensates generated since they surprisingly do not have an adverse effect on isophorone nitrile synthesis and on the contrary yields and selectivities in fact increase in their presence. A further advantage is that the energy demand over the reaction as a whole may be reduced when separation, especially distillation, of the isophorone-rich fraction may be omitted.

The present invention thus preferably provides a process for producing isophorone nitrile wherein a mixture which comprises the valuable substances isophorone and at least one keto- and optionally hydroxyl-containing hydrocarbon having a molecular weight of 178-276 g/mol and is obtainable by a process comprising the following steps a) to d) in that order
a) subjecting acetone to a base-catalysed aldol condensation to afford isophorone,
b) hydrolyzing the valuable stream from the aldol condensation,
c) performing a phase separation of the hydrolysate and
d) freeing the organic phase deriving from the phase separation from organic components having a molar mass of <126 g/mol
   with hydrogen cyanide to afford isophorone nitrile. In this preferred embodiment the mixture comprising the valuable substances isophorone and at least one keto- and optionally hydroxyl-containing hydrocarbon having a molecular weight of 178-276 g/mol is the organic phase deriving from the organic phase separation after separation of the organic components having a molar mass of <126 g/mol. Especially in this embodiment according to the invention the product mixture deriving from the organic phase of the phase separation after the hydrolysis is reacted directly and without further purification with hydrogen cyanide to afford isophorone nitrile after the relatively low-energy removal of organic components having a molar mass of <126 g/mol.

The base-catalysed aldol condensation reaction a) from acetone to isophorone is preferably carried out in a liquid phase reaction. However, isophorone may in principle also be produced from acetone in the gas phase or in supercritical acetone.

It is preferable when the acetone is converted to isophorone in the liquid phase at temperatures of 100-250° C., preferably 150-250° C., particularly preferably 180-250° C., and in a pressure range of 5-50 bar, preferably 10-50 bar, particularly preferably of 20-50 bar, wherein the reported values may be combined with one another as desired.

In a gas phase reaction the acetone is converted to isophorone within the employed reactor by catalytic reaction preferably at temperatures in the range from 100° C. to 400° C., preferably 200-400° C.

In the supercritical range the acetone is converted to isophorone within the employed reactor by catalytic reaction preferably at temperatures in the range from 250° C. to 350° C. and a pressure range of 50 to 200 bar.

The catalysts employed as bases may be homogeneous or heterogeneous catalysts. In the liquid phase it is preferable to employ a homogeneous catalyst. In the gas phase it is preferable to employ a heterogeneous catalyst. For the reaction in the supercritical range it is possible to use either homogeneous or heterogeneous catalysts.

The condensation of acetone to form isophorone is preferably carried out under base catalysis. Especially in the liquid phase the employed base is preferably an alkali metal hydroxide solution. The basic catalyst is yet more preferably selected from the group consisting of NaOH and KOH.

The content of base, especially of alkali metal hydroxide solution, more preferably NaOH or KOH, is preferably <1% by weight, preferably <0.5% by weight, particularly preferably <0.2% by weight, based on the reaction composition. It is very particularly preferable to employ the catalyst NaOH in an amount of 0.015% to 0.05% by weight.

The water concentration employed results inter alia from the recycle streams from the workup processes. The water content of the reaction mixture, based on the total amount of liquid, is preferably not more than 30% by weight, preferably not more than 20% by weight.

The acetone content in the reaction mixture, based on the total amount of liquid, is preferably not more than 60% by weight, more preferably not more than 70% by weight, particularly preferably not more than 75% by weight.

The reaction may be carried out in any desired reactors, preferably in tubular reactors, stirred tanks, stirred tank cascades, fixed bed reactors, pressure distillation reactors/reactive distillation reactors, microstructured reactors, loop reactors or in combinations of the recited reactors.

The term “pressure distillation reactor” is to be considered synonymous with apparatuses in which a reactive distillation is performed. Reactive distillation is adequately described in the specialist literature.

After performing the aldol condensation the valuable stream is preferably separated from the reaction mixture. The reaction mixture is preferably worked up and separated into the individual components. These comprise not only isophorone but also low boilers (especially acetone, diacetone alcohol and mesityl oxide), higher condensation products (overcondensates) of acetone (especially xylitones and isoxylitones), water and catalyst.

Fractionation into the components may in principle be performed using any separation methods, in particular distillation, flash evaporation, crystallization, extraction, sorption, permeation, phase separation or combinations thereof, continuously or batchwise, in one or more stages. Separation is preferably carried out by distillation in one or more apparatuses.

The distillation may be performed spaced apart from the isophorone synthesis (reaction) or be carried out in one apparatus. The separation of the individual fractions is preferably carried out by means of a reactive distillation, preferably in a reactive distillation column.

The separation is particularly preferably performed spaced apart from the isophorone synthesis (reaction) in a reactive distillation column having a sidestream takeoff.

The separation is preferably performed so as to obtain the following three fractions:
i) A fraction of unreacted acetone, water and low boilers (especially diacetone alcohol and mesityl oxide)
ii) A fraction in which especially colour-imparting substances are enriched.
iii) A fraction comprising especially isophorone, more highly condensed products, water and catalyst, the so-called valuable stream. This fraction is subsequently subjected to a hydrolysis.

Fraction i) is preferably condensed and then recycled to the reactor for reaction. In a further preferred embodiment said fraction is withdrawn as a vapour stream containing essentially acetone, water and low boilers, essentially diacetone alcohol and mesityl oxide, condensed and returned to the reactor with the inputs acetone, water and optionally catalyst.

In a preferred embodiment fraction ii) is withdrawn as a sidestream of the distillation column, preferably of a reactive distillation column, optionally neutralized and worked up further. This fraction is purified further and the materials present recycled into the process.

The workup may employ any standard separation methods, for example distillation, flash evaporation, crystallization, extraction, sorption, permeation, phase separation or combinations of thereof. The purification may be performed continuously or batchwise, in one or more stages. The purification is preferably achieved by distillation. The purification is particularly preferably achieved by a combination of neutralization or extraction and subsequent distillation, preferably in a reactive distillation column. The worked-up phase comprising the valuable products selected from isophorone, high-boilers and optionally catalyst is preferably sent to the hydrolysis. A further obtained phase composed of valuable products essentially containing acetone, diacetone alcohol and mesityl oxide is preferably recycled into the reaction. Any residues obtained are sent for thermal recovery.

Fraction iii) is preferably obtained in the bottom of the distillation column and can be withdrawn therefrom to obtain the valuable stream.

The valuable stream from the aldol condensation is then preferably b) hydrolysed, i.e. subjected to hydrolysis. The aim of the hydrolysis is partial or complete conversion of byproducts present in the valuable stream into isophorone, acetone or other usable valuable products. The hydrolysis may be performed in all standard reactors, distillation columns or combinations thereof. It is preferable to perform the hydrolysis as a reactive distillation in which the low boilers formed, essentially containing acetone, diacetone alcohol and mesityl oxide, are removed directly from the hydrolysis zone and recycled to the reaction and are thus no longer available for secondary reactions in the hydrolysis.

The hydrolysis of fraction iii) may very particularly preferably be performed in an apparatus by means of reactive distillation, preferably in a reactive distillation column, with simultaneous separation of the reaction mixture into fractions i) to iii), with the result that the products formed are appropriately fractionated, and fraction iii) hydrolysed, simultaneously.

The hydrolysis and the distillative removal may optionally also be carried out in one apparatus with the isophorone synthesis (reaction).

The hydrolysis may be performed with any mixing ratios of the organic components with water, with or without catalyst. The water concentration in the hydrolysis is 30.1-99.9% by weight based on the total mass of the constituents present. In the case of homogeneous catalysis, the catalyst used in the hydrolysis is preferably that which is also used in the aldol condensation. It is preferable to select catalyst concentrations of 0.001-10% by weight, particularly preferably of 0.05-1% by weight, based on the total mass of the composition. The pressure in the hydrolysis reactor is 1-200 bar, preferably 20-60 bar. The hydrolysis is particularly preferably performed at least at the pressure also prevailing in the isophorone synthesis step (reaction). The temperature of the hydrolysis is 100-300° C., preferably 210-260° C. When using a reactive distillation column it is particularly preferable when a temperature or a temperature profile is established according to the boiling temperatures in the column bottom and at the individual separation or reaction stages.

The hydrolysis may be performed in one or more apparatuses, in one or more stages.

Once it has further preferably been discharged from the hydrolysis reactor of the reactive distillation column and cooled, the obtained hydrolysate is then preferably subjected to a phase separation c).

The phase separation affords a substantially organic fraction i) and a substantially aqueous fraction ii). In the case of homogeneous catalysis of the aldol condensation the aqueous fraction ii) also contains the catalyst. The phase separation may employ customary phase separation vessels with or without internals. The phase separation is carried out at a temperature between 0-200° C., preferably at 0-100° C., particularly preferably at 20-70° C., and a pressure of 1-150 bar, preferably 20-60 bar, particularly preferably at the pressure at which the hydrolysis is also carried out.

The substantially organic phase i) comprising the target product isophorone has previously been optionally neutralized and purified by customary methods to obtain an isophorone with the desired purity and colour stability. Any standard separation methods, especially distillation, flash evaporation, crystallization, extraction, sorption, permeation, phase separation or combinations thereof, may have been employed. The purification may have been performed continuously or batchwise, in one or more stages, under pressure or under vacuum. The purification has preferably been achieved by distillation. The purification has particularly preferably been achieved by a combination of neutralization or extraction and subsequent distillation.

Contrary to the former approach the organic phase deriving from the phase separation is now only freed of organic components having a molar mass of <126 g/mol before the remainder of the organic phase is reacted with hydrogen cyanide to afford isophorone nitrile. The separation of organic components having a molar mass of <126 g/mol is preferably carried out by a single-stage process.

The organic phase i) is preferably freed of organic components having a molar mass of <126 g/mol by thermal separation of organic components. This is more preferably effected by distillation or rectification at a pressure of 60-150 mbar, a bottoms temperature of 130-170° C. and a tops temperature of 70-120° C., wherein isophorone and all components having a greater molar mass than isophorone (especially the hydrocarbons having a molecular weight of 178-276 g/mol and high boilers having a molar mass >276 g/mol) remain in the bottoms. In a very particularly preferred embodiment these bottoms together with added isophorone may be directly employed for the process according to the invention for producing isophorone nitrile.

The fraction containing the components having a molecular weight of <126 g/mol may be discarded. However, in a preferred embodiment it is once again subjected to a phase separation after addition of water. The resulting aqueous phase is then sent for aldol condensation, hydrolysis or wastewater treatment (see paragraph below) and the obtained organic fraction sent for thermal recovery.

The substantially aqueous fraction ii) may be sent to a wastewater purification. This effects separation of the water of reaction as the primary constituent and optionally of the catalyst from any still-dissolved organic components, for example isophorone, acetone and more highly condensed products. The wastewater purification is preferably performed in one or more distillation columns.

According to the invention the mixture comprising the valuable substances isophorone and at least one keto- and optionally hydroxyl-containing hydrocarbon having a molecular weight of 178-276 g/mol is reacted with hydrogen cyanide to afford isophorone nitrile. The reaction of the valuable substance-containing mixture with hydrogen cyanide is preferably performed under base catalysis since the basic catalysts supply free cyanide ions for the 1,4-addition reaction.

Preferred catalysts may be selected from the group consisting of basic alkali metal or alkaline earth metal compounds, onium compounds, organic bases and phase-transfer catalysts. The catalyst may very particularly preferably be selected from the group consisting of basic alkali metal or alkaline earth metal compounds. The catalyst may yet more preferably be selected from the group consisting of alkali metal alkoxides, oxides, hydroxides, carbonates, peroxides and cyanides and alkaline earth metal alkoxides, oxides, hydroxides, peroxides and cyanides.

The reaction with hydrogen cyanide (HCN) may be performed either in solution or solventlessly.

The catalysis may be catalysed homogeneously or heterogeneously. In the case of homogeneous catalysis the catalyst and the reactants are in the same phase. In heterogeneous catalysis the catalyst and the reactants are in different phases.

The process according to the invention preferably employs homogeneous base catalysis. To this end it is very particularly preferable to employ alkali metal alkoxides, especially sodium methoxide, as catalysts.

The hydrogen cyanide may be used in pure form or else as a mixture with stabilizers. Suitable stabilizers include all compounds known to those skilled in the art for this purpose, for example phosphoric acid, sulfur dioxide and oxalic acid. It is preferable to use phosphoric acid, sulfur dioxide or mixtures thereof as stabilizers for the hydrocyanic acid. The proportion of stabilizers is typically in the range from 0.1 to 2% by weight based on the employed hydrocyanic acid, preferably in the range from 0.5 to 1% by weight.

The reaction may be undertaken in the presence or absence of inert solvents. It is particularly preferable to employ isophorone in molar excess based on the employed hydrocyanic acid and not to employ any external solvent.

The process according to the invention is generally performed so as to employ an excess of isophorone since this achieves a higher selectivity for isophorone nitrile. The isophorone/HCN molar ratio is preferably >1:1, more preferably 19:1 to 1.5:1, preferably 3:1 to 1.5:1.

The catalyst concentration based on the employed isophorone amount is in the range from 0.03% to 20% by weight, preferably in the range from 0.03% to 1% by weight.

It is preferable when the total amount of the isophorone is initially charged and brought to the desired reaction temperature before the hydrogen cyanide is added in the presence of the catalyst.

The HCN addition is preferably carried out so as to ensure a homogeneous distribution of the reactants (isophorone and HCN). The HCN addition may be carried out in a manner that is customary and known to those skilled in the art, preferably via static mixers, pumps or atomization.

The reaction temperatures are typically in the range from 130 to 225° C., preferably in the range from 135° C. to 195° C. and very particularly preferably in the range from 140° C. to 175° C.

The reaction is typically performed at pressures of 0.01 to 10 bar, preferably 1 to 5 bar, particularly preferably at standard pressure (atmospheric pressure).

Within the aforementioned reaction parameters the HCN is preferably added such that a sufficiently low concentration of HCN results and thus a high selectivity and a high conversion to isophoronenitrile may be achieved.

It is preferable when the concentrations of unconverted, free HCN and the total concentration of cyanide ions (sum of free HCN and cyanide bound in the form of cyanohydrins of isophorone and isophoronenitrile) are determined and the reaction conditions adapted so as to obtain an optimized selectivity with respect to IPN. Said cyanide ion concentrations are preferably determined by titration.

The process according to the invention may preferably be performed continuously, discontinuously or semicontinuously, especially continuously.

In a preferred embodiment the reaction procedure is performed in a stirred tank, a stirred tank cascade, a recirculating reactor, a flow tube, one or more fixed bed reactors or a column according to the employed catalyst.

It is preferable when excess isophorone is removed by distillation and advantageously reused. To this end the separated isophorone is mixed with fresh isophorone and recycled into the reaction reactor. The isophorone nitrile formed is separated from possible secondary components by distillative workup and may be further employed.

The isophorone nitrile obtainable by the process may be used for the synthesis of isophoronediamine by aminating hydrogenation. The present invention thus also provides for the use of the isophorone nitrile obtainable by the process according to the invention for the synthesis of isophoronediamine.

Unless otherwise stated all reported percentages (%) are percentages by mass.

The examples recited below describe the present invention by way of example without any intention to limit the invention, the scope of application of which is apparent from the entirety of the description and the claims, to the embodiments specified in the examples.

### EXAMPLES

### Example 1

1. Production of Isophorone Nitrile from Crude Isophorone and HCN in a Batch Process

The conversion of crude isophorone to isophorone nitrile was carried out in a 1 L reactor fitted with a stirrer and a dropping funnel. 464 g of crude isophorone (from hydrolysis after phase separation and separation of material of molar mass <126 g/mol, 89.5% by weight isophorone, 8.8% by weight mass range 178-276 g/mol, 1.6% by weight unidentified components, see table 1) was initially charged in the reactor and temperature-controlled to 20° C. A mixture of 18.8 g of crude isophorone (from hydrolysis after phase separation and separation of material of molar mass <126 g/mol, 89.5% by weight isophorone, 8.8% by weight mass range 178-276 g/mol, 1.6% by weight unidentified components, see following table) and 40 g of HCN were added to the dropping funnel at −6° C. The reactor was heated to 175° C., the catalyst sodium methoxide (30% by weight solution in methanol) was added to the isophorone initially charged in the reactor and the catalyst initial charge was washed with 5 g of crude isophorone (from hydrolysis after phase separation and separation of material of molar mass <126 g/mol, 89.5% by weight isophorone, 8.8% by weight mass range 178-276 g/mol, 1.6% by weight unidentified components, see table 1). Addition of the HCN/crude isophorone mixture was subsequently commenced. The addition is carried out at 175° C. and standard pressure over 30 minutes. After complete addition the reaction mixture was subjected to further stirring at 175° C. under reflux for a further 30 minutes. This resulted in an isophorone conversion of 37.6% (calculated based on the starting concentration of isophorone in the crude isophorone) and a selectivity for isophorone nitrile of 92.8% (based on the starting concentration of isophorone in the crude isophorone).

2. Production of Isophorone Nitrile from a Mixture of Pure Isophorone and Crude Isophorone (1:1) and HCN in a Batch Process

The conversion of isophorone to isophorone nitrile was carried out in a 1 L reactor fitted with a stirrer and a dropping funnel. 464 g of isophorone (mixture of pure and crude isophorone 1:1, 94.6% by weight isophorone, 4.5% by weight mass range 178-276 g/mol, 0.9% by weight unidentified components, see table 1; the crude isophorone is the same as in example 1) was initially charged in the reactor and temperature-controlled to 20° C. A mixture of 18.8 g of isophorone (mixture of pure and crude isophorone 1:1, 94.6% by weight isophorone, 4.5% by weight mass range 178-276 g/mol, 0.9% by weight unidentified components, see table 1; the crude isophorone is the same as in example 1) and 40 g of HCN were added to the dropping funnel at −6° C. The reactor was heated to 175° C., the catalyst sodium methoxide (30% by weight solution in methanol) was added to the isophorone initially charged in the reactor and the catalyst initial charge was washed with 5 g of isophorone (mixture of pure and crude isophorone 1:1, 94.6% isophorone, 4.5% by weight mass range 178-276 g/mol, 0.9% by weight unidentified components, see table 1; the crude isophorone is the same as in example 1). Addition of the HCN/isophorone mixture was subsequently commenced. The addition is carried out at 175° C. and standard pressure over 30 minutes. After complete addition the reaction mixture was subjected to further stirring at 175° C. under reflux for a further 30 minutes. This resulted in an isophorone conversion of 38.7% (calculated based on the starting concentration of isophorone in the crude isophorone) and a selectivity for isophorone nitrile of 93.5% (based on the starting concentration of isophorone in the crude isophorone).

3. Production of Isophorone Nitrile from Pure Isophorone and HCN in a Batch Process

The conversion of isophorone to isophorone nitrile was carried out in a 1 L reactor fitted with a stirrer and a dropping funnel. 464 g of isophorone (after fine distillation, 99.7% by weight isophorone, 0.1% by weight mass range 178-276 g/mol, 0.2% by weight unidentified components, see table 1) was initially charged in the reactor and temperature-controlled to 20° C. A mixture of 18.8 g of isophorone (after fine distillation, 99.7% by weight isophorone, 0.1% by weight mass range 178-276 g/mol, 0.2% by weight unidentified components, see table 1) and 40 g of HCN were added to the dropping funnel at −6° C. The reactor was heated to 175° C., the catalyst sodium methoxide (30% by weight solution in methanol) was added to the isophorone initially charged in the reactor and the catalyst initial charge was washed with 5 g of isophorone (after fine distillation, 99.7% by weight isophorone, 0.1% by weight mass range 178-276 g/mol, 0.2% by weight unidentified components, see table 1).

Addition of the HCN/isophorone mixture was subsequently commenced. The addition is carried out at 175° C. and standard pressure over 30 minutes. After complete addition the reaction mixture was subjected to further stirring at 175° C. under reflux for a further 30 minutes. This resulted in an isophorone conversion of 40.7% (calculated based on the starting concentration of isophorone in the crude isophorone) and a selectivity for isophorone nitrile of 78.3% (based on the starting concentration of isophorone in the crude isophorone).

TABLE 1

| | | Exp. 2 | |
|---|---|---|---|
| | Exp. 1 | (1:1 mixture of | Exp. 3 |
| Material | (crude IP) | pure and crude IP) | (pure IP) |
| Reactant composition | | | |
| Isophorone | 89.5 | 94.6 | 99.7 |
| Mass range 178-276 g/mol | 8.8 | 4.5 | 0.1 |
| Unidentified component | 1.6 | 0.9 | 0.2 |
| Product composition | Mass fraction [%] based on GC | | |
| Isophorone | 51.9 | 55.9 | 59.0 |
| Isophorone nitrile | 34.9 | 36.2 | 31.9 |
| Mass range 178-276 g/mol | 7.5 | 5.6 | 6.8 |
| Unidentified component | 5.7 | 2.3 | 2.4 |
| Conversion | 37.6 | 38.7 | 40.7 |
| Selectivity | 92.8 | 93.5 | 78.3 |

1. Process for producing isophorone nitrile comprising reacting a mixture comprising the valuable substances
a) isophorone and
b) at least one keto- and optionally hydroxyl-containing hydrocarbon having a molecular weight of 178-276 g/mol
with hydrogen cyanide to afford isophorone nitrile.

2. Process according to claim 1,
wherein the mixture comprises at least two different keto- and optionally hydroxyl-containing hydrocarbons having a molecular weight of 178-276 g/mol.

3. Process according to claim 1,
wherein at least one of the keto- and optionally hydroxyl-containing hydrocarbons having a molecular weight of 178-276 g/mol has a structure selected from one of the following structural formulae (I) to (IX):

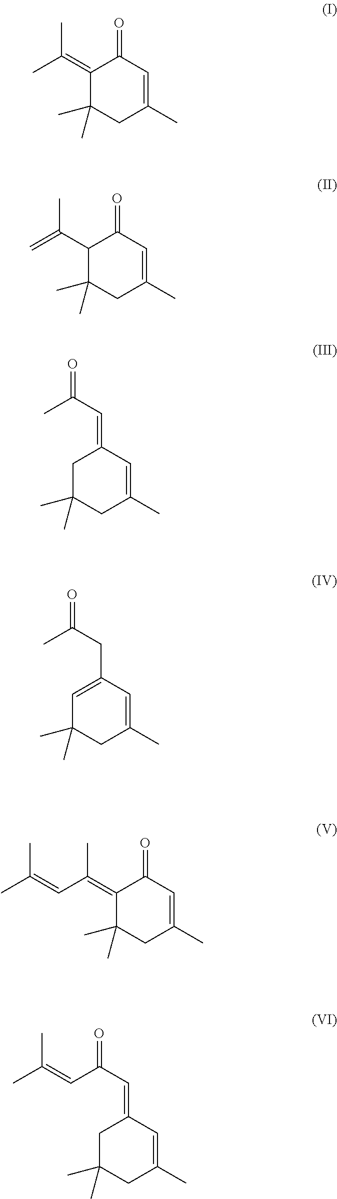

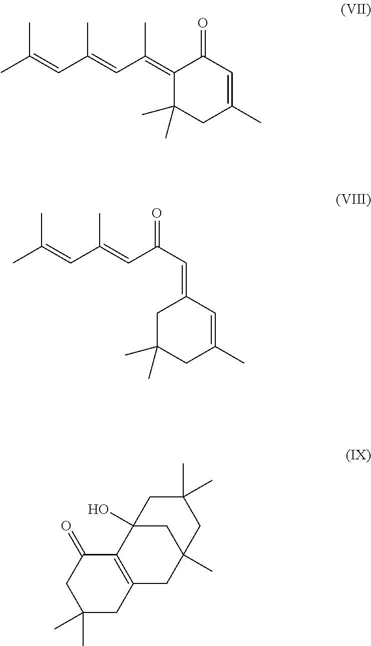

4. Process according to claim 1,
wherein the mass fraction of keto- and optionally hydroxyl-containing hydrocarbons having a molecular weight of 178-276 g/mol in the valuable substance mixture, based on the total mass of isophorone and keto- and optionally hydroxyl-containing hydrocarbons having a molecular weight of 178-276 g/mol, is between 1% and 50% by weight.

5. Process according to claim 1,
wherein the mixture which comprises the valuable substances isophorone and at least one keto- and optionally hydroxyl-containing hydrocarbon having a molecular weight of 178-276 g/mol is obtainable by a process comprising the following steps a) to d) in that order
a) subjecting acetone to a base-catalysed aldol condensation to afford isophorone,
b) hydrolyzing the valuable stream from the aldol condensation,
c) performing a phase separation of the hydrolysate and
d) freeing the organic phase deriving from the phase separation from organic components having a molar mass of <126 g/mol.

6. Process according to claim 5,
wherein the aldol condensation a) is performed in the liquid phase at a temperature of 100-250° C. and a pressure of 5-50 bar.

7. Process according to claim 5,
wherein the hydrolysis of the valuable stream b) is performed at a temperature of 210-260° C. and a pressure of 20-60 bar.

8. Process according to claim 5,
wherein the phase separation is carried out at a temperature of 0-100° C. and a pressure of 20-60 bar.

9. Process according to claim 5,
wherein the organic phase i) is freed of organic components having a molar mass of <126 g/mol by thermal separation of organic components.

10. Process according to claim 5,
wherein the reaction with hydrogen cyanide is performed under base catalysis.

11. Process according to claim 10,
wherein the reaction with hydrogen cyanide is carried out under homogeneous catalysis with at least one alkali metal alkoxide as catalyst.
